(19) United States
(12) Patent Application Publication
(10) Pub. No.: US 2007/0292501 A1
(43) Pub. Date: Dec. 20, 2007

(54) CHEWABLE SOFT GELATIN CAPSULES

(76) Inventor: Ronald G. Udell, Beverly Hills, CA (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/757,789

(22) Filed: Jun. 4, 2007

Related U.S. Application Data
(60) Provisional application No. 60/810,917, filed on Jun. 5, 2006.

(51) Int. Cl. A61K 9/64

(52) U.S. Cl. 424/456

(57) ABSTRACT

The present invention is directed to compositions and methods of delivery of fill materials containing active agents, optionally dissolved or suspended in a suitable carrier, encapsulated in a chewable soft gelatin capsule.

### CROSS-REFERENCE TO RELATED APPLICATION(S)

This application claims the benefit of U.S. Provisional Application No. 60/810,917, filed Jun. 5, 2006, entitled “Chewable Soft Gelatin Capsule” by Michael Fantuzzi, the entire contents of which is incorporated herein by reference.

### FIELD OF THE INVENTION

The present invention relates to delivery of a fill material encapsulated within a chewable soft gelatin capsule.

### BACKGROUND OF THE INVENTION

Encapsulation of active agents, such as pharmaceuticals, vitamins, antioxidants, and the like, can be accomplished with hard gelatin capsules or soft gelatin capsules. Soft gelatin compositions used to prepare the shell of the capsule are not conducive to chewing to release the active agent. Most of the soft gelatin shells provide a gummy, unpleasant tasting, intractable mass in the mouth when chewed.

Sometimes the individual or mammal has a difficult time swallowing traditional pills that are either hard or soft, due to age, gag reflex, diminished throat muscle strength due to stroke, and other ailments. Sometimes the pill is not palatable to the subject due to size, taste, or other factors.

There are instances where the subject is in immediate need of rapid application of the active agent, such as an angina attack. The shell of the soft gelatin capsule must dissolve rapidly upon chewing so as to release the active agent.

Therefore, there is a need in the art for an improved methodology to deliver active agents to an individual or mammal, such as a dog, in need thereof.

### BRIEF SUMMARY OF THE INVENTION

The present invention pertains to chewable soft gelatin capsules containing a fill material that includes an active agent. The chewable soft gelatin shell includes at least gelatin, a plasticizer other than xylitol, xylitol and water. The fill material encapsulated within the chewable soft gelatin shell, includes an active agent, and optionally, an acceptable carrier.

In one embodiment the chewable soft gelatin capsule consists essentially of gelatin, a plasticizer other than xylitol, i.e., glycerin, xylitol and water.

In another embodiment, the chewable soft gelatin shell does not include a starch acetate.

In still another embodiment, the chewable soft gelatin shell does not include two types of gelatin.

In one aspect, the shell can further include maltitol, wherein the maltitol is present at a weight percentage of the total weight of the shell of less than about 4 weight percent.

In another aspect, the shell and/or fill material can include a coloring agent and/or a flavoring agent.

In still another aspect, the present invention also includes packaged formulations of the soft gelatin chewable capsules.

The present invention provides the advantage of a chewable soft gelatin capsule for individuals who might not otherwise easily take a pill or tablet. This is especially true for the elderly and small children where swallowing can be problematic. Likewise, animals, such as dogs, cats and horses, often do not readily take “hard” pills and tablets. The present invention solves these issues by use of a chewable capsule while delivering the beneficial active agent to the subject.

In one aspect, the shell is hydrogenated starch hydrolysate free. This is advantageous for those subjects that are allergic to starch based products.

In another aspect, the shell contains less than 4% by weight of hydrogenated starch hydrolysate. Again, utilization of a very minimal amount of a hydrogenated starch hydrolysate is advantageous for those subjects that are allergic to starch based products.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### DETAILED DESCRIPTION

The present invention provides chewable soft gelatin capsules that contain an encapsulated fill material of an active agent and, optionally, a suitable carrier. In one aspect, the chewable soft gelatin shell includes at least gelatin, a plasticizer other than xylitol, xylitol and water. The carrier is especially optional where the active agent is a liquid that does not dissolve the chewable soft gelatin shell.

The phrase “sufficient quantity of a carrier suitable to solubilize an active agent” is therefore intended to mean that that amount of a carrier that will dissolve or suspend the active agent under a given set of conditions, generally, those at ambient temperature. This determination should be understood by one skilled in the art and can be determined by methods known in the art, such as by solubility studies.

One type of suitable carrier is a monoterpene. The term “monoterpene” as used herein, refers to a compound having a 10-carbon skeleton with non-linear branches. A monoterpene refers to a compound with two isoprene units connected in a head-to-end manner. The term “monoterpene” is also intended to include “monoterpenoid”, which refers to a monoterpene-like substance and may be used loosely herein to refer collectively to monoterpenoid derivatives as well as monoterpenoid analogs. Monoterpenoids can therefore include monoterpenes, alcohols, ketones, aldehydes, ethers, acids, hydrocarbons without an oxygen functional group, and so forth.

It is common practice to refer to certain phenolic compounds, such as eugenol, thymol and carvacrol, as monoterpenoids because their function is essentially the same as a monoterpenoid. However, these compounds are not technically “monoterpenoids” (or “monoterpenes”) because they are not synthesized by the same isoprene biosynthesis pathway, but rather by production of phenols from tyrosine. However, common practice will be followed herein. Suitable examples of monoterpenes include, but are not limited to, limonene, pinene, cintronellol, terpinene, nerol, menthane, carveol, S-linalool, safrol, cinnamic acid, apiol, geraniol, thymol, citral, carvone, camphor, etc. and derivatives thereof. For information about the structure and synthesis of terpenes, including terpenes of the invention, see Kirk-Othmer Encyclopedia of Chemical Technology, Mark, et al., eds., 22:709-762 3d Ed (1983), the teachings of which are incorporated herein in their entirety.

In particular, suitable limonene derivatives include perillyl alcohol, perillic acid, cis-dihydroperillic acid, trans-dihydroperillic acid, methyl esters of perillic acid, methyl esters of dihydroperillic acid, limonene-2-diol, uroterpenol, and combinations thereof.

Other suitable carriers useful in the fill material include but are not limited to, for example, fatty acids, esters and salts thereof, that can be derived from any source, including, without limitation, natural or synthetic oils, fats, waxes or combinations thereof. Moreover, the fatty acids can be derived, without limitation, from non-hydrogenated oils, partially hydrogenated oils, fully hydrogenated oils or combinations thereof. Non-limiting exemplary sources of fatty acids (their esters and salts) include seed oil, fish or marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, flaxseed oil, evening primrose oil, jojoba, tallow, beef tallow, butter, chicken fat, lard, dairy butterfat, shea butter or combinations thereof.

Specific non-limiting exemplary fish or marine oil sources include shellfish oil, tuna oil, mackerel oil, salmon oil, menhaden, anchovy, herring, trout, sardines or combinations thereof. In particular, the source of the fatty acids is fish or marine oil (DHA or EPA), soybean oil or flaxseed oil. Alternatively or in combination with one of the above identified carrier, beeswax can be used as a suitable carrier, as well as suspending agents such as silica (silicon dioxide).

It should be understood that the term “comprising” (or comprises) includes the more restrictive terms consisting of and consisting essentially of.

The term “mammal” is recognized in the art and includes any of various warm-blooded vertebrate animals of the class Mammalia, including humans, characterized by a covering of hair on the skin and, in the female, milk-producing mammary glands for nourishing the young. Examples include horses, dogs, cats, cows, pigs, sheep, goats, and the like.

The term “animal” is also recognized in the art and pertains to those mammals other than humans. Examples are those above, including horses, dogs, cats, cows, pigs, sheep, goats, and the like.

The term “canine” is recognized in the art and generally refers to dogs.

The chewable soft gelatin formulations of the invention are considered dietary supplements useful to provide an effective amount of an active agent to the individuals or animals in need thereof.

The term “effective amount” is intended to mean that amount useful to achieve a desired effect. For example, an effective amount of a vitamin would be the recommended daily requirement. As another example, an effective amount of an antibiotic would be that amount required to help diminish or eradicate the bacterial infection in the subject. Of course, this treatment may take several days and multiple doses of the active agent within the chewable soft gelatin capsule during each day's treatment.

Alternatively, the formulations of the invention are also considered to be nutraceuticals. The term “nutraceutical” is recognized in the art and is intended to describe specific chemical compounds found in foods that may prevent disease. Many of the active agents set forth throughout the specification are considered to be neutraceuticals.

The formulations, the fill materials, of the invention include one or more active agents that can be supplements to an individual's diet. Suitable active agents can include vitamins and biologically-acceptable minerals. Non-limiting examples of vitamins include vitamin A, B vitamins, vitamin C, vitamin D, vitamin E, vitamin K and folic acid. Non-limiting examples of minerals include iron, calcium, magnesium, potassium, copper, chromium, zinc, molybdenum, iodine, boron, selenium, manganese, derivatives thereof or combinations thereof. These vitamins and minerals may be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

Vitamin(s), if present, are present in the composition of the invention in an amount ranging from about 5 mg to about 500 mg. More particularly, the vitamin(s) is present in an amount ranging from about 10 mg to about 400 mg. Even more specifically, the vitamin(s) is present from about 250 mg to about 400 mg. Most specifically, the vitamin(s) is present in an amount ranging from about 10 mg to about 50 mg. For example, B vitamins are in usually incorporated in the range of about 1 milligram to about 10 milligrams, i.e., from about 3 micrograms to about 50 micrograms of B12. Folic acid, for example, is generally incorporated in a range of about 50 to about 400 micrograms, biotin is generally incorporated in a range of about 25 to about 700 micrograms and cyanocobalamin is incorporated in a range of about 3 micrograms to about 50 micrograms.

Mineral(s), if present, are present in the composition of the invention in an amount ranging from about 25 mg to about 1000 mg. More particularly, the mineral(s) are present in the composition ranging from about 25 mg to about 500 mg. Even more particularly, the mineral(s) are present in the composition in an amount ranging from about 100 mg to about 600 mg.

Other exemplary active agents that can be incorporated into the fill material of the chewable soft gelatin capsules include, without limitation, phospholipids, L-camitine, starches, sugars, fats, antioxidants, amino acids, proteins, flavorings, coloring agents, hydrolyzed starch(es) and derivatives thereof or combinations thereof.

As used herein, the term “phospholipid” is recognized in the art, and refers to phosphatidyl glycerol, phosphatidyl inositol, phosphatidyl serine, phosphatidyl choline, phosphatidyl ethanolamine, as well as phosphatidic acids, ceramides, cerebrosides, sphingomyelins and cardiolipins.

L-camitine is recognized in the art and facilitates transport of materials through the mitochondrial membrane. L-carnitine is an essential fatty acid metabolism cofactor that helps to move fatty acids to the mitochondria from the cytoplasm.

As used herein, the term “antioxidant” is recognized in the art and refers to synthetic or natural substances that prevent or delay the oxidative deterioration of a compound. Exemplary antioxidants include tocopherols, flavonoids, catechins, superoxide dismutase, lecithin, gamma oryzanol; vitamins, such as vitamins A, C (ascorbic acid) and E and beta-carotene; natural components such as camosol, camosic acid and rosmanol found in rosemary and hawthorn extract, proanthocyanidins such as those found in grapeseed or pine bark extract, glutathione, alpha-lipoic acid and green tea extract.

The term “flavonoid” as used herein is recognized in the art and is intended to include those plant pigments found in many foods that are thought to help protect the body from cancer. These include, for example, epi-gallo catechin gallate (EGCG), epi-gallo catechin (EGC) and epi-catechin (EC).

Polycosinol, red yeast rice, gugulipid, pantethine, garlic, chromium, carnitine, artichoke leaf, green tea, Gymnema sylvestre, grape seed extract, pine bark extract, ginseng and silymarin are additional active agents that can be used in the present formulations to treat one or more of ailments, including lowering cholesterol, indicated within the present specification.

Policosanol (polycosanol) consists of a mixture of fatty alcohols derived from waxes of sugar cane (the main source of policosanol) yams, and beeswax. The main ingredient of policosanol is octanosol. These active substances act to lower cholesterol levels by several mechanisms that include blocking the formation of cholesterol in the liver.

Not to be limited by theory, it is believed that the alcohols in policosanol act on cholesterol metabolism in the liver, but at a different part of the metabolic pathway than statins. Many animal studies with policosanol demonstrate a cholesterol lowering effect, and more recently human studies have suggested that LDL cholesterol can be reduced to a degree similar to that achieved with statins, and that HDL cholesterol can be increased by as much as 10-25% (an effect difficult to achieve with statins). Policosanol also reduces the platelet aggregation (i.e., the “stickiness” of platelets, the blood elements that promote blood clotting).

Generally, between about 5 milligrams and about 50 milligrams of policosanol is included in a composition of the invention, in particular, between about 10 milligrams and about 30 milligrams, and more particularly between about 10 milligrams and about 20 milligrams on a weight basis.

Typically a composition is provided that includes about 10 milligrams of policosanol. Generally, two, three, four or more dosages of the composition are taken over the course of a day to provide between about 10 and 20 milligrams of policosanol.

Red yeast rice (extract) is an Asian dietary staple made by fermenting red yeast (Monascus purpureus) on rice and is recognized as a cholesterol-lowering agent. This is due in part to a careful fermentation process that yields statins, compounds known to reduce cholesterol levels.

As a substance, red yeast rice extract has a number of heart-healthy benefits. It helps reduce total cholesterol levels, lower levels of LDL cholesterol, increase levels of HDL cholesterol, and lower levels of triglycerides. Not to be limited by theory, it appears that red yeast rice (and its extracts) accomplish this by restricting the liver's production of cholesterol. The compound responsible for this effect, mevinolin, is chemically identical to the cholesterol lowering compound known as lovastatin, which is sold as the prescription drug Mevacor. Additionally, unsaturated fatty acids in red yeast rice extract are also believed to be beneficial, possibly by lowering triglycerides.

Generally, between about 300 milligrams and about 1200 milligrams of red yeast rice extract is included in a composition of the invention, in particular, between about 400 milligrams and about 1000 milligrams, and more particularly between about 500 milligrams and about 800 milligrams on a weight basis.

Typically a composition is provided that includes about 300 milligrams of red yeast rice extract. Generally, two, three, four or more dosages of the composition are taken over the course of a day to provide between about 600 and 1200 milligrams of read yeast rice extract.

Gugulipid is an extract from the mukul myrrh tree (Commiphora mukul) that is native to India. It is a purified extract standardized for compounds known as guggulsterones. The active compounds in gugulipid believed to be responsible for the cholesterol-lowering properties are two steroids: E- and Z-guggulsterone. Several clinical studies have shown gugulipid has an ability to lower both cholesterol and triglyceride levels.

The mechanism of action for gugulipid's cholesterol lowering action is its ability to increase the liver's ability to breakdown cholesterol. The dosage of gugulipid is based on its guggulsterone content.

Related benefits are its ability to prevent plaque from forming in arterial walls, which can lead to heart attacks if unchecked. Moreover, it acts as an anti-coagulant by inhibiting blood platelets from clumping together, and, therefore, affords protection against blood clots.

Gugulipid also harbors antioxidant properties; antioxidants scavenge free radicals, which are highly reactive substances that damage cells, leading to premature disease and aging.

Generally, between about 100 milligrams and about 700 milligrams of gugulipid is included in a composition of the invention, in particular, between about 200 milligrams and about 600 milligrams, and more particularly between about 250 milligrams and about 500 milligrams on a weight basis.

Typically a composition is provided that includes about 250 milligrams of gugulipid. Generally, two, three, four or more dosages of the composition are taken over the course of a day to provide between about 500 and 1000 milligrams of gugulipid.

Garlic can help reduce cholesterol. Garlic is a proven antioxidant and this property helps to prevent LDLs from being oxidized. In this way the cholesterol build up is believed to be reduced by garlic.

Generally, between about 200 milligrams and about 500 milligrams of garlic is included in a composition of the invention, in particular, between about 250 milligrams and about 400 milligrams, and more particularly between about 300 milligrams and about 350 milligrams on a weight basis.

milligrams of green tea is included Typically a composition is provided that includes about 250 milligrams of garlic. Generally, two, three, four or more dosages of the composition are taken over the course of a day to provide between about 500 and 4000 milligrams of garlic.

Pantethine is a combination of pantothenic acid (vitamin B-5) and beta mercaptoethylamine. Panteteine is the precursor to coenzyme A, the critical starting point in the Krebs energy production cycle.

Pantethine is the disulfide dimer of pantetheine, the 4′-phosphate derivative of which is an intermediate in the conversion of the B vitamin pantothenic acid to coenzyme A. Pantethine is found naturally in small quantities in most forms of life, and therefore, in food sources. Pantethine has lipid-lowering effects. Pantethine is also known as D-bis(N-pantothenyl-beta-aminoethyl)disulfide and (R)—N,N′-[dithiobis(ethyleneimino-carbonylethylene]bis(2,4-dihydroxy-3,3-dimethylbutyramide). Its molecular formula is C₂₂H₄₂N₄O₈S₂ and its molecular weight is 554.73 daltons.

Pantethine has been found to decrease serum levels of total cholesterol, low-density lipoprotein cholesterol (LDL-C), apolipoprotein B and triglycerides. It has also been found to increase high-density lipoprotein cholesterol (HDL-C) and apolipoprotein Al levels. In isolated hepatocytes, pantethine has been shown to inhibit both cholesterol and fatty acid synthesis. It is believed that pantethine, by acting as a precursor of coenzyme A, may enhance the beta-oxidation of fatty acids.

Generally, between about 200 milligrams and about 500 milligrams of pantethine is included in a composition of the invention, in particular, between about 200 milligrams and about 400 milligrams, and more particularly between about 250 milligrams and about 300 milligrams on a weight basis.

Typically a composition is provided that includes about 500 milligrams of pantethine. Generally, two, three, four or more dosages of the composition are taken over the course of a day to provide between about 500 and 1000 milligrams of pantethine.

Chromium lowers total and LDL cholesterol levels and raises HDL cholesterol levels in the blood, particularly in people with high cholesterol.

Chromium is generally utilized as a complex. There are various chromium complexes available that can be included in the compositions of the invention. These include, but are not limited to, chromium chloride, chromium picolinate, chromium chloride, chromium nicotinate, and high-chromium yeast.

For example, chromium polynicotinate, in particular, is a trace mineral that helps regulate carbohydrate metabolism. Since all carbohydrates are reduced in the body into simple glucose, chromium polynicotinate provides the go-between action by “plugging” serum glucose from the bloodstream directly to the muscle cell. Chromium is a necessary component for carbohydrate metabolism, glucose regulation, and energy production.

Chromium polynicotinate is a mineral utilized in the regulation of blood sugar. It is involved in the metabolism of glucose and is a key component for energy. The ability to maintain stable blood sugar levels is often jeopardized by diets that are often high in white flour, refined sugar and junk food. Chromium polynicotinate facilitates and/or stimulates the metabolism of sugar, fat and cholesterol in the body, as well as the function of insulin.

Chromium picolinate can lead to significant improvements in body composition resulting from fat loss, particularly for individuals who may not be as aggressive in making lifestyle changes such as reducing caloric intake or increasing their physical activity. It is believed that chromium picolinate's positive effect on body composition is through its ability to improve insulin utilization, thereby reducing fat deposition and resulting in improving entry of glucose and amino acids into muscle cells.

Generally, between about 200 micrograms and about 600 micrograms of chromium is included in a composition of the invention, in particular, between about 200 micrograms and about 400 micrograms, and more particularly between about 250 micrograms and about 300 micrograms on a weight basis.

Typically a composition is provided that includes about 200 micrograms of chromium. Generally, two, three, four or more dosages of the composition are taken over the course of a day to provide between about 200 and 600 micrograms of chromium.

Artichoke (cynara scolymus) has a number of beneficial effects, and has been used in jaundice and liver insufficiency as well as for cholesterol reduction. It is considered that artichoke inhibits oxidation of low density lipoprotein and reduces cholesterol biosynthesis. Active components of artichoke are cynarine and luteolin.

The leaves of the artichoke contain a high content of pharmacologically active ingredients, including three essential groups consisting of caffeeolyquinic acid (CCS), flavonoids and bitter substances. Within these groups are constituents such as caffeic acid, chlorogenic acid, cynarine (1,5-dicaffeolyquinic acid), luteolin, and the glycosides scolymoside and cynaroside. Among the most important of the CCS are the 1,3-Di-O—CCS, choloregenic acid and the Cynarin.

Generally, between about 100 milligrams and about 300 milligrams of artichoke is included in a composition of the invention, in particular, between about 200 milligrams and about 300 milligrams, and more particularly between about 220 milligrams and about 250 milligrams on a weight basis.

Typically a composition is provided that includes about 300 milligrams of artichoke. Generally, two, three, four or more dosages of the composition are taken over the course of a day to provide between about 500 and 1200 milligrams of artichoke.

Green tea is an herb (Camellia sinensis). Green tea originated in China, Japan and other parts of Asia. The leaf of the plant is used in creating the extract that is potent and bioflavonoid-rich. This herb is used primarily for its free-radical scavenging capabilities.

Green tea is prepared by picking, lightly steaming and allowing the leaves to dry. The active constituents in green tea are a family of polyphenols (catechins) and flavonols that possess potent antioxidant activity. Tannins, large polyphenol molecules, form the bulk of the active compounds in green tea, with catechins comprising nearly 90%. Several catechins are present in significant quantities; epicatechin (EC), epigallocatechin (EGC), epicatechin gallate (ECG) and epigallocatechin gallate (EGCG). EGCG makes up about 10-50% of the total catechin content and appears to be the most powerful of the catechins with antioxidant activity about 25-100 times more potent than vitamins C and E.

Generally, between about 100 milligrams and about 300 in a composition of the invention, in particular, between about 200 milligrams and about 300 milligrams, and more particularly between about 220 milligrams and about 250 milligrams on a weight basis.

Typically a composition is provided that includes about 100 milligrams of green tea. Generally, two, three, four or more dosages of the composition are taken over the course of a day to provide between about 100 and 300 milligrams of green tea.

Gymnema sylvestre, also known as Gurmarbooti or Gurmar, is a woody climbing plant that grows in the tropical forests of central and southern India. Cholesterol reducing activity is attributed to members of a family of substances called gymnemic acids.

Gymnemic acids, the active ingredients, are thought to have a gradual blood sugar lowering effect that may result from enhancing the overall function and health of pancreatic insulin releasing cells and reducing insulin resistance. As a liquid, gymnema blocks the absorption of dietary fats into the bloodstream.

Generally, between about 100 milligrams and about 300 milligrams of Gymnema sylvestre is included in a composition of the invention, in particular, between about 200 milligrams and about 300 milligrams, and more particularly between about 220 milligrams and about 250 milligrams on a weight basis.

Typically a composition is provided that includes about 300 milligrams of Gymnema sylvestre. Generally, two, three, four or more dosages of the composition are taken over the course of a day to provide between about 500 and 1000 milligrams of Gymnema sylvestre.

Grape seed extract includes specialized flavonoids called oligomeric proanthocyanidins (OPCs). Studies suggest grape seed helps improve blood circulation, prevent atherosclerosis, lowers blood pressure and decreases low density lipoprotein cholesterol levels and increases high density lipoprotein levels.

The OPCs are chemically known as flavonoids or polyphenols, which can differ substantially based on their polymer arrangement. For example, polyphenols can exist in single (monomers), double (dimers), triple (trimers), quadruple (tetramers) and even longer cyanidin chains (tannins). Any chain length from 2-7 or so is referred to as an oligomer and longer chains are generally referred to as polymers.

Generally, between about 100 milligrams and about 300 milligrams of grape seed extract is included in a composition of the invention, in particular, between about 200 milligrams and about 300 milligrams, and more particularly between about 220 milligrams and about 250 milligrams on a weight basis.

Typically a composition is provided that includes about 100 milligrams of grape seed extract. Generally, two, three, four or more dosages of the composition are taken over the course of a day to provide between about 100 and 300 milligrams of grape seed extract.

Pine bark extract can be included in the compositions of the invention to reduce low density lipoproteins and to help strengthen blood vessel walls. Pine bark extract is also known as French Marine Pine Bark Extract, French Maritime Pine Bark Extract, Leucoanthocyanidins, OPC, Oligomeric Proanthocyanidins, PCO, Pine Bark, Pinus maritima, Pinus pinaster, Procyandiol Oligomers, Procyanodolic Oligomers, Pycnogenol, and Pygenol.

Generally, between about 100 milligrams and about 300 milligrams of pine bark extract is included in a composition of the invention, in particular, between about 200 milligrams and about 300 milligrams, and more particularly between about 220 milligrams and about 250 milligrams on a weight basis.

Typically a composition is provided that includes about 100 milligrams of pine bark extract. Generally, two, three, four or more dosages of the composition are taken over the course of a day to provide between about 100 and 300 milligrams of pine bark extract.

Panax ginseng is also called ginseng, Korean ginseng, schinsent, or ninjin. Ginseng is an adaptogen that has been used to lower cholesterol, balance the metabolism, increase energy levels, and stimulate the immune system.

Ginseng is characterized by the presence of ginsenoside. Ginsenosides are a class of steroid-like compounds, triterpene saponins, found exclusively in ginseng.

Generally, between about 25 milligrams and about 200 milligrams of ginseng is included in a composition of the invention, in particular, between about 50 milligrams and about 150 milligrams, and more particularly between about 75 milligrams and about 100 milligrams on a weight basis.

Typically a composition is provided that includes about 50 milligrams of ginseng. Generally, two, three, four or more dosages of the composition are taken over the course of a day to provide between about 25 and 200 milligrams of ginseng.

Extracts of Milk Thistle protect against liver toxins through the action of antihepatotoxic (liver protectant) compounds commonly referred to as silymarin. Silymarin has been shown to consist of a large number of flavonolignans, including silybin, isosilybin, dehydrosilybin, silydianin and silychristin. Silymarin, and component silybin, function as antioxidants, protecting cell membranes from free-radical-mediated oxidative damage. Both silymarin and silybin protect red blood cell membranes against lipid peroxidation and hemolysis (breaking down of the red blood cells) caused by certain red blood cell poisons and have also been found to reduce total serum cholesterol.

Generally, between about 25 milligrams and about 200 milligrams of silymarin is included in a composition of the invention, in particular, between about 50 milligrams and about 150 milligrams, and more particularly between about 75 milligrams and about 100 milligrams on a weight basis.

Typically a composition is provided that includes about 100 milligrams of silymarin. Generally, two, three, four or more dosages of the composition are taken over the course of a day to provide between about 240 and 500 milligrams of silymarin.

Another active agent can be a polymethoxylated flavone (PMF). The PMF can be one or more of limocitrin, limocitrin derivatives, quercetin and quercetin derivatives, including but not limited to limocitrin-3,7,4′-trimethylether (5-hydroxy-3,7,8,3′,4′-pentamethoxyflavone); limocitrin-3,5,7,4′-tetramethylether (3,5,7,8,3′,4′-hexamethoxyflavone); limocitrin-3,5,7,4′-tetraethylether (8,3′-dimethoxy-3,5,7,4′-hexamethoxyflavone); limocitrin-3,7,4′-trimethylether-5-acetate; quercetin tetramethylether (5-hydroxy-3,7,3′,4′-tetramethoxyflavone); quercetin-3,5-dimethylether-7,3′,4′-tribenzyl ether; quercetin pentamethyl ether (3,5,7,3′,4′-pentamethoxyflavone); quercetin-5,7,3′,4′-tetramethylether-3-acetate; and quercetin-5,7,3′,4′-tetramethylether (3-hydroxy-5,7,3′,4′-tetramethoxyflavone); and the naturally occurring polymethoxyflavones: 3,5,6,7,8,3′,4′-heptan-ethoxyflavone; 5-desmethylnobiletin (5-hydroxy-6,7,8,3′,4′-pentamethoxyflavone); tetra-O-methylisoscutellarein (5,7,8,4′-tetramethoxyflavone); 5-desmethylsinensetin (5-hydroxy-6,7,3′,4′-tetramethoxyflavone); and sinensetin (5,6,7,3′,4′-pentamethoxyflavone).

Pine nut oil promotes stimulation of a protein called cholecystokinin (CCK). This protein, produced in the small intestine and also present in the brain, is produced in the duodenum after eating and sends a “full” feeling to the brain. At the same time, CCK slows the rate of stomach emptying, further enhancing the feeling of satiety.

Pinolenic acid, an active ingredient isolated from the genus Pinus, is a triple-unsaturated fatty acid which is a positional isomer of a more widely known gamma-linolenic acid [GLA]) and is found exclusively in pine nut oil. The structure of pinolenic acid is

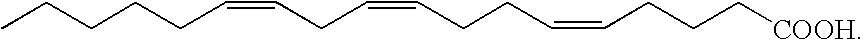

This fatty acid is present in all 140 varieties of pine nuts (and their oil) in quantities ranging from 0.1 to more than 20 percent. However, the richest known source of pinolenic acid is the oil pressed from the seeds of the Siberian pine (Pinus Sibirica) or from the Korean pine (Pinus koraiensis).

Suitable pine nut oil extracts that contains pinolenic acid are commercially available from, for example, Lipid Nutrition, Durkee Road 24708, Channahon, Ill., USA under the trademark PinnoThin™ or Siberian Tiger Naturals, Inc., 81 Glinka Road, Cabot, Vt. USA.

Derivatives of pinolenic acid include esters, such as methyl and ethyl esters, mono-, di-, and triglycerides and acceptable salts of the carboxylic acid. Esters can be prepare by transesterification of the carboxylic acid by techniques known in the art. Acceptable salts include alkali, alkaline, and ammonium salts and the like.

Generally, between about 15 milligrams and about 1500 milligrams of pine nut oil can be included in a composition of the invention, in particular, between about 300 milligrams and about 750 milligrams, and more particularly between about 500 milligrams and about 700 milligrams, i.e. 500 milligrams on a weight basis.

Typically a composition is provided that includes between about 350 milligrams and about 400, i.e., 375 milligrams of pine nut oil. Generally, two, three, four or more dosages of the composition are taken over the course of a day to provide between about 1000 milligrams and about 2000 milligrams of the pine nut oil, e.g., between about 1250 milligrams and about 1500 milligrams.

Active ingredients from Hoodia or Trichocaulon can be isolated by an extraction procedure by extracting sap from the plant and then spray-drying the sap. Alternatively, solvent extraction procedures can be employed. In either case, fractionation of the initial extract, e.g. by column chromatography, can be used to generate an extract with enhanced activity.

The extract can be prepared from plant material such as the stems and roots of plants of the genus Hoodia or the genus Trichocaulon that grow in the arid regions of southern Africa. The plant extract is generally obtained from one of the species: Trichocaulon piliferum; Trichocaulon officinale; Hoodia currorii; Hoodia gordonii; and Hoodia lugardii.

Extracts from Hoodia or Trichocaulon provide steroidal glycosides, which appear to fool the brain into thinking the stomach is “full” and act as appetite suppressants. One such steroidal glycoside of importance is known as P57 or P57AS3. P57 is the compound 3-0-[-β-D-thevetopyranosyl-(1->4)-β-D-cymaropyranosyl-(1->4)-β-D-cymaropyranosyl]-12β-0-tigloyloxy-14-hydroxy-14β-pregn-50-en-20-one (C₄₇H₇₄O15 M+878). The identification and isolation of P57 and Hoodia and Trichocaulon extracts are found in U.S. Pat. No. 6,376,657, the contents of which are incorporated herein by reference in their entirety for all purposes.

Derivatives suitable for use in the compositions of the invention include those formula disclosed throughout U.S. Pat. No. 6,376,657, the contents of which are incorporated herein by reference in their entirety for all purposes.

Hoodia and/or Trichocaulon extracts are commercially available from various suppliers such as Stella Laboratories, Paramus, N.J.

Generally, between about 50 milligrams and about 250 milligrams of Hoodia extract can be included in a composition of the invention, in particular, between about 100 milligrams and about 200 milligrams, and more particularly between about 150 milligrams and about 175 milligrams on a weight basis.

Typically a composition is provided that includes between about 50 milligrams and about 250 milligrams of Hoodia extract, i.e., Hoodia gordonii. Generally, two, three, four or more dosages of the composition are taken over the course of a day to provide between about 50 milligrams and about 1000 milligrams of the Hoodia gordonii, e.g., between about 100 milligrams and about 500 milligrams.

Conjugated linoleic acid is still another acceptable active agent. The term “conjugated linoleic acid” is intended to include any conjugated linoleic acid or octadecadienoic fatty acid, including all positional and geometric isomers of linoleic acid with two conjugated carbon-carbon double bonds at any position in the molecule. Suitable examples of CLA include cis- and trans isomers (“E/Z isomers”) of the following positional isomers: 2,4-octadecadienoic acid, 4,6-octadecadienoic acid, 6,8-octadecadienoic acid, 7,9-octadecadienoic acid, 8,10-octadecadienoic acid, 9,11-octadecadienoic acid, 10,12 octadecadienoic acid and 11,13 octadecadienoic acid. As used herein, “CLA” encompasses a single isomer, a selected mixture of two or more isomers, and a non-selected mixture of isomers obtained from natural sources, as well as synthetic and semisynthetic CLA. The term is intended to include non-naturally occurring isomers of CLA.

CLA is an omega 6 oil. Suitable sources of CLA include, for example, sunflower oil, corn oil, or safflower oil. Typically, the oils provide a CLA content of between about 70 and about 90% (by weight), more particularly between about 75 and about 85%, and even more particularly, between about 78 and about 84% by weight.

It is believed that CLA reduces body fat by enhancing insulin sensitivity so that fatty acids and glucose can pass through muscle cell membranes and away from fat tissue. This results in an improved muscle to fat ratio. Compelling evidence indicates that CLA can promote youthful metabolic function and reduce body fat.

The term “isomerized conjugated linoleic acid” refers to a CLA synthesized by chemical methods (e.g., aqueous alkali isomerization, non-aqueous alkali isomerization, or alkali alcoholate isomerization).

The term “conjugated linoleic acid derivative” refers to any compound or plurality of compounds containing conjugated linoleic acids or derivatives thereof. Examples include fatty acids, alkyl esters, triglycerides of conjugated linoleic acid as well as nutritionally acceptable salts thereof.

It should be understood that “triglycerides” of CLA contain CLA at any or all of three positions (e.g., SN-1, SN-2, or SN-3 positions) on the triglyceride backbone. Accordingly, a triglyceride containing CLA can contain any of the positional and geometric isomers of CLA.

“Esters” of CLA include any and all positional and geometric isomers of CLA bound between the carboxylic acid portion to an alcohol or any other chemical group, including, but not limited to physiologically acceptable, naturally occurring alcohols (e.g., methanol, ethanol, propanol). Therefore, an ester of CLA or esterified CLA may contain any of the positional and geometric isomers of CLA.

The phrase “non-naturally occurring isomers” of CLA includes, but is not limited to c11,t13; t11,c13; t11,t13; c11,c13; c8,t10; t8,c10; t8,t10; c8,c10; and trans-trans isomers of octadecadienoic acid, and does not include t10,c12 and c9,t11 isomers of octadecadienoic acid. “Non-naturally occurring isomers” may also be referred to as “minor isomers” of CLA as these isomers are generally produced in low amounts when CLA is synthesized by alkali isomerization.

The term, “low impurity” CLA refers to CLA compositions, including free fatty acids, alkylesters, and triglycerides, which contain less than 1% total 8,10 octadecadienoic acids, 11,13 octadecadienoic acids, and trans-trans octadecadienoic acids.

The abbreviation, “c” encompasses a chemical bond in the cis orientation, and “t” refers to a chemical bond in the trans orientation. If a positional isomer of CLA is designated without a “c” or a “t”, then that designation includes all four possible isomers. For example, 10,12 octadecadienoic acid encompasses c10,t12; t10,c12; t10,t12; and c10,c12 octadecadienoic acid, while t10,c12 octadecadienoic acid or CLA refers to just the single isomer.

Salts of CLA include salts as described previously.

A suitable CLA for preparation of the compositions of the invention, is known as TONALIN®, and is available from Cognis Nutrition & Health, LaGrange, Ill., USA.

Generally, between about 250 milligrams and about 500 milligrams of CLA can be included in a composition of the invention, in particular, between about 250 milligrams and about 400 milligrams, and more particularly between about 300 milligrams and about 350 milligrams on a weight basis.

Typically a composition is provided that includes between about 250 and about 500 milligrams of the CLA. Generally, two, three, four or more dosages of the composition are taken over the course of a day to provide between about 500 and about 4000 milligrams of CLA, e.g., about 2000 milligrams per day.

Starch blockers are known in the art and the term is intended to include, but is not limited to, alpha amylase inhibitors, alpha-glucoside inhibitors and glucosidase inhibitors. In particular, the present invention includes starch blockers based on derivatives from white kidney beans (Phaseolis vulgaris). Phaseolamin is a partially-purified protein extract of white kidney beans that binds to alpha-amylase enzymes, which are responsible for the digestive breakdown of starch. It has been proposed that phaseolamin inhibits the alpha-amylase breakdown of starch by non-competitively binding the enzyme to prevent the hydrolysis of the alpha-1,4-glycosidic linkages in the starch molecule.

Alpha-glucosidase is an enzyme that breaks disaccharides into their respective monosaccharide units. Alpha-glucosidase inhibitors prevent the enzyme from performing this function. A wide variety of alpha-glucosidase inhibitors are known and any suitable inhibitor can be used in the compositions and methods of the present invention. Examples of suitable alpha-glucosidase inhibitors include, but are not limited to, voglibose (see U.S. Pat. No. 6,200,958 to Odaka et al.), acarbose (see U.S. Pat. No. 5,643,874 to Bremer et al.), and touchi extract. Touchi is a traditional Chinese food derived from soybeans. Touchi is prepared by first steaming and then fermenting soybeans with Aspergillus species bacteria

Alpha-amylase is an enzyme that functions to break the alpha-1,4-glycosidic linkages present in starch. This breaks the complex starch molecule into smaller units, such as disaccharides, that can be further digested by other enzymes, such as alpha-glucosidase. Alpha-amylase inhibitors prevent the enzyme from hydrolyzing the alpha-1,4-glycosidic bond, and therefore prevent the breakdown of starch. A wide variety of alpha-amylase inhibitors are known, and any suitable inhibitor can be used in the compositions and methods of the present invention. Examples of suitable alpha-amylase inhibitors include, but are not limited to, an inhibitor extracted from wheat (see U.S. Pat. No. 3,950,319 to Schmidt et al.), Amylostatin-A (see U.S. Pat. No. 4,010,258 to Murao), and phaseolamin.

In one aspect, the compositions of the invention include the alpha-amylase inhibitor phaseolamin. Phaseolamin is an extract of the white kidney bean (Phaseolus vulgaris). The extract is water-soluble and rich in protein content. Phaseolamin is readily available from numerous commercial suppliers. Phaseolamin PHASEOLAMIN 2250®, available from Pharmachem Laboratories of Kearny, N.J. and also known as PHASE 2®, is a standardized extract particularly well-suited for inclusion in the compositions according to the present invention. This phaseolamin demonstrates a high ability to block alpha-amylase activity.

Generally, between about 125 milligrams and about 350 milligrams of starch blocker can be included in a composition of the invention, in particular, between about 125 milligrams and about 333 milligrams, and more particularly between about 150 milligrams and about 250 milligrams on a weight basis.

Typically a composition is provided that includes between about 125 and about 333 milligrams of the starch blocker. Generally, two, three, four or more dosages of the composition are taken over the course of a day to provide between about 250 and about 3000 milligrams of the starch blocker, e.g., between about 1000 milligrams and about 2000 milligrams.

Ceramides are also suitable active agents. The term “ceramide” is recognized in the art and is related to sphingolipids that are present in cell structures. Ceramides are also notably present in the plant world and in particular in wheat, rice, soya, millet, olive and spinach. Alternatively, ceramides can also be synthesized.

The term ceramide, therefore, includes lipids composed of the sphingosine family, such as sphinganine, 4 hydroxy-sphinganine or phytosphingosine, which are bonded to a fatty acid or fatty acid derivative via their amine functional group. The term ceramide thus includes all ceramides, of synthetic or natural origin (vegetable, animal or human) optionally substituted, for example, by a sugar such as mono- or polyglucosylceramides.

The term “processed ceramide” refers to a ceramide or ceramide analog that is isolated from its non-naturally occurring state, such as the naturally occurring ceramide found in wheat or rice. That is to say, processed ceramides include the ceramide oils, powders, gums, etc. isolated from treated natural sources that contain ceramide, such as wheat, oryza, rice etc. (pressed, extracted, distilled, fractionated, etc.).

The ceramides of the stratum corneum are composed of 6 chromatographically distinct fractions having a different polarity according to the degree of unsaturation (which may be zero) or of hydroxylation of their chains, their length and their number. The ceramides are categorized according to their chemical configuration as class I, II, III, IV, V, VIa and VIb. Their chemical configuration is in particular provided in the document “Ceramides, Key Components for Skin Protection,” by R. D. Petersen, Cosmetics & Toiletries, vol. 107, February 1992, p. 45-49 and the document in EJD, No. 1, vol. 1, October 1991, Review article, p. 39-43, “Skin Ceramides: Structure and Function,” by M. Kerscher, the contents of which are incorporated herein in their entirety.

As stated above, ceramides used in the present invention are amide derivatives in which fatty acids are attached to the amine groups of sphingosine, phtyosphingosine, or sphinganine, exemplified by the following structures:

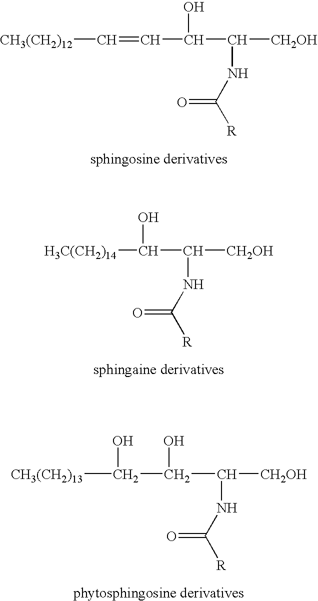

wherein R is a C₆-C₂₅ unsaturated or saturated carbon chain which has one or more double bonds or a C₆-C₂₅ unsaturated or saturated carbon chain with a hydroxy group at the alpha or beta position.

Plants contain structures, which are chemically similar to human ceramides. These ceramide products can help in creating the protective barrier in the epidermis. Supplementation with an oral ceramide composition replaces the components lost with aging. The moisturizing effect directly comes from the ceramides being carried directly to the stratum corneum via the blood. This direct method improves the functionality of the agent and produces results not seen in cosmetic topical applications.

The term ceramide also includes oryza ceramide (derived from oryza sativa), a brown extract from rice bran or rice germ that contains large amounts of glycosphingolipid. Both glycosphingolipids of rice bran and those derived from animal sources include an sphingoid base with a fatty acid amide linkage, with the terminal hydroxyl group being substituted by glucose (or another sugar moiety). Oryza ceramides are commercially available from companies such as Oryza Oil & Fat Co. Ltd., Japan. Likewise, phyto-ceramides are extracted from Konjac-tuber (Amorphophallus konjac) and are glycosylated ceramides. Konjac-tuber ceramide is commercially available from Unitika Ltd, Kyoto, Japan. Another suitable ceramide source is “OptiPure®” Ceramide from Chemco Industries, Los Angeles, USA.

For example, in one embodiment, ceramide is derived from a special fraction of plant based product. The ceramide itself is extracted with ethanol at ninety nine degrees Celsius. The flour fraction is incorporated into the ethanol under a rapid mechanical stirring for two hours. The whole separation is transferred into a Buchner filter. The process then moves into a clarification of the filtrate, which consists of wheat lipids and ethanol. After large insoluble particles are removed, the extraction solvent is distilled into a reactor distillatory. Separation of polar liquids and apolar liquids take place. The oil is then mixed in hot water with an emulsor during 30 minutes and the mixture is allowed to settle overnight. The polar lipids and particularly, the sphingolipids and glycosphingolipids form a stable emulsion in water. During the recovery phase, apolar liquids have a density inferior to water and rise to the surface of the mixture; so water containing polar lipids can be collected by gravity filtration. The water fraction is then freeze dried, resulting in a powder which is crushed and micronized, packed into hermetically sealed bags with a desiccant.

Therefore, it should be understood that the terms “ceramide” or “ceramide analog thereof” are inclusive of all types of ceramides described throughout the present specification, including sugar substituted (glycosylated) and those as are known in the art.

Feverfew is yet another suitable active agent. The herb, which is used in the present invention, is known correctly and variously as Chrysanthemum parthenium, Tanecetum parthenium and Matricania parthenium. All known plants having the characteristics, are therefore included in this application. Of these plants, Tanacetum parthenium (also known as Chrysanthenum parthenium and commonly known as Feverfew, featherfoil, flirtwort and Bachelor's Buttons) has been put forward as a herbal medicament or nutraceutical.

The extracts of Tanacetum parthenium that contain various volatile oils having mono- and/or sesquiterpene components, flavonoids, tannins, and pyrethrin, as well as terpenoids of the family of sesquiterpene lactones known as germacranolides, guaianolides and eudesmanolides are considered part of the invention. These latter compounds are characterized by an alpha-unsaturated gamma-lactone structure (sequiterpene lactones, STLs). In particular the STLs include parthenolide, 3-beta-hydroxy-parthenoide, costunolide, 3-beta-hydroxy-costunolide, artemorin, 8-alpha-hydroxy-estaflatin and chrysanthemonin, all of which are considered to be parthenolide analogs for the purposes of this invention.

Parthenolide is an active component of Feverfew extracts. Parthenolide, represents about 85% of the STL content in Feverfew and is the portion of the leaf believed to be responsible for Feverfew's anti-migraine and nutraceutical properties. Parthenolide (1 aR,4E,7aS,10aS,10bS)-2,3,6,7,7a,8,10a,10b-Octahydro-1a,5-dimethyl-8-methylene-oxireno[9,10]cyclodeca[1,2-b]furan-9(1aH)-one, depicted below, includes an epoxide and a lactone ring:

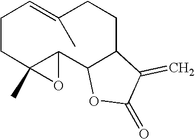

Feverfew extracts, containing parthenolides, have been isolated by various methods. However, the parthenolides, which are considered to be the active components of the extracts, are generally unstable and lose a portion of their nutraceutical activity within a year. This is possibly due to instability of the epoxide ring and/or from hydrolysis of the lactone ring. Additionally, common extraction techniques only provide Feverfew extracts that have about 1.0% (generally 0.7%) or less parthenolide(s) on a weight basis of total extract.

Various extraction procedures useful to isolate an extract that includes parthenolides include those described in U.S. Pat. Nos. 4,758,433, 5,384,121, 6,224,875, 6,479,080 and 6,23,768, the contents of which are incorporated herein by reference in their entirety. These extraction procedures generally require the use of a polar organic solvent for example acetonitrile, methanol, ethanol, isopropanol, ether, ethyl acetate, acetone or mixtures thereof. Ethanol is the preferred solvent, since it is the least toxic with regards to the residues being left in the final product.

Not to be limited by theory, it is believed the Feverfew extract can be “stabilized” by treatment of the extract with an organic acid, in particular, an organic acid having two or more carboxylic acid moieties. In certain embodiments, the carboxylic acid is a salt. Suitable carboxylic acid containing moieties that are useful as “stabilizers” include, for example, adipic acid, pyruvic acid, gallic acid, tartaric acid and citric acid. Suitable salts include a metal ion, e.g., an alkali metal ion, an alkaline earth ion, or an aluminum or calcium ion; or coordinates with an organic base such as ethanolamine, diethanolamine, triethanolamine, N-methylglucamine, morpholine, piperidine, dimethylamine, diethylamine and the like. Also included are salts of amino acids such as arginates and the like, and salts of organic acids like glucurmic or galactunoric acids and the like (see, e.g., Berge et al., 1977, J. Pharm. Sci. 66:1-19). In a particular embodiment, the salt of the carboxylic acid is tricalcium citrate. Generally, the ratio of stabilizer to FeverFew extract (on a dry weight basis) is in a range of between about 0.05 to 1 to about 1:1, more particularly from between about 0.2 to 1 to about 0.5 to 1, and also, 0.3 to 1 to about 0.6 to 1.

Still other active agents include hyaluronic acid, corosolic acid (banaba leaf extract), analgesics, calcium channel blockers, fatty acids, anti-Parkinson agents, bronchodilators, sedatives, anti-HCV compounds, beta-blockers, antibacterials, antidepressants, anti-inflammatory agents, cerebral stimulants, antidiabetics, decongestants, muscle relaxants, cholesterol lowering agents, and the like.

Active agents also include antitussive compounds such as dextromethorphan, detromethorphan hydrobromide, noscapine, carbetapentane citrate, chlorphedianol hydrochloride and the like.

Active agents further include sedating antihistamines such as chlorphenramine, phenidamine, doxylamine, phenyl-oxamine, diphenhydramine, promethazine, triprolidine, hydroxyzine, meclinzine, cyproheptadine, azatadine their salts and mixtures thereof.

Suitable non-sedating antihistamines include fexofenadine, terfenadine, astemizole, loratadine and cetirizine.

Suitable decongestants include phenylephrine, phenylpropanolamine, pseudoephedrine, ephedrine, their salts and mixtures thereof.

Nonsteroidal anti-inflammatory agents (NSAIDS) cam also be incorporated in fill materials of the present invention. Suitable NSAIDS include ibuprofen, ketoprofen, acetylsalicylic acid, ketoprofen, aproxen, naprosyn, meclomen, indomethicin and mixtures thereof.

Suitable analgesics include acetaminophen.

Suitable H²-antagonists useful in the fill materials of the present invention include famotidine, ranitidine, cimetidine and mixtures thereof.

Useful antibiotics, antibacterials and bactericidals include erythromycin, cephalosporin, tetracyclines, penicillin, amoxycillin, clathromycin and mixtures thereof.

Useful anti-convulsants include phenyltoin and thosuximide.

The wide variety of active agents useful herein include their acid addition salts. Both organic and inorganic salts may be used and exemplary acid salts include the hydrochloride, hydrobromide, orthophosphate, benzoate, maleate, tartrate, succinate, citrate, salicylate sulfate, acetate and mixtures thereof.

It should be understood that two or more of the active agents can be combined in the fill material.

Coloring agents useful in the fill material are generally considered oil-soluble so as to disperse/dissolve appropriately in the optional carrier if present. Suitable coloring agents include cochineal, annatto, caramel, carmine oil, carotenoids, xanthins, cryptoxanthin, paprika, carrot oils, chlorophyllin, carob extract, tumeric power/oil, anthocyanins, lycopene, astaxanthin, etc. and can be formulated in the mixture from about zero weight percent to about 5 weight percent, more specifically between about 0.5 and about 3 weight percent and in particular about 2 weight percent.

Flavoring agents useful in the fill material include cinnamon, cinnamon oil, citric acid, lemon oil, orange oil, nutmeg oil, peppermint oil, rose oil, spearmint, spearmint oil, strawberry oil, bacon flavor, barbeque flavors, beef, beef fat, cheese flavors, such as cheddar, nacho, mozzarella, romano, parmesan, chicken, clam, egg, fat, fish, ham, hot dog, lamb, lard, liver, lobster, meat and cheese blend, oyster, pizza, pork, pork liver, prawn, savory, seafood, smoked salmon, steak, taco, tallow and teriyaki and can be formulated in the mixture from about zero weight percent to about 5 weight percent, more specifically between about 0.5 and about 5 weight percent and in particular about 4 weight percent.

Chewable soft gel or chewable soft gelatin capsules can be prepared, for example, without limitation, by dispersing the formulation, as described above in an appropriate vehicle (e.g. rice bran oil, monoterpene and/or beeswax) to form a mixture. This mixture, the fill material, is then encapsulated with the chewable gelatin based film using technology and machinery known to those in the soft gel industry. The industrial units so formed are then dried to constant weight. Typically, the weight of the capsule is between about 100 to about 2500 milligrams and in particular weigh between about 1500 and about 1900 milligrams, and more specifically can weigh between about 1500 and about 2000 milligrams.

For example, when preparing chewable soft gelatin shells, the chewable shell can include gelatin, generally a plasticizer other than xylitol, xylitol and water. The filling of the chewable soft gelatin capsule is liquid (principally a carrier) and can include, apart from the active agent, a hydrophilic matrix. The hydrophilic matrix, if present, is a generally a polyethylene glycol having an average molecular weight of from about 200 to 1000. Alternatively, the matrix can include sorbitol and/or sorbitol special. Further ingredients are optionally thickening agents. In one embodiment, the hydrophilic matrix includes polyethylene glycol having an average molecular weight of from about 200 to 1000, 5 to 15% glycerol, and 5 to 15% by weight of water. The polyethylene glycol can also be mixed with propylene glycol and/or propylene carbonate.

Suitable plasticizers, other than xylitol, include glycerol (glycerin), sorbitol, polyglycerol, non-crystallizing solutions of sorbitol, sorbitol special, glucose, fructose and glucose syrups with different equivalents and mixtures thereof. The inclusion of glycerol provides a more chewable product.

The term “fill material” is intended to mean a substantially water-free material (generally less than about 10% water) which includes at least one active compound, and optional amounts of co-solvents, buffers, surfactants, thickeners, and the like as described throughout the specification with reference to the formulations. The fill material may be of solid, semi-solid, gel, or liquid form, so long as it is compatible with the chewable soft gelatin encapsulation, so that it does not substantially degrade the chewable soft gelatin shell.

In another embodiment, the chewable soft gel capsule is prepared from gelatin, glycerin, xylitol, water and various additives. Typically, the percentage (by weight) of the gelatin is between about 15 and about 50 weight percent, in particular between about 20 and about 40 weight percent, more particularly between about 20 and about 35 weight percent, and more specifically about 35 weight percent. The formulation includes between about 10 and about 50 weight percent glycerin, in particular between about 15 and about 40 weigh percent, more particularly between about 20 and about 30 weight percent and more specifically about 31 weight percent glycerin.

A portion of the chewable capsule is typically water. The amount varies from between about 5 weight percent and about 50 weight percent, in particular between about 10 and about 40 weight percent, more particularly between about 10 and about 25 weight percent, and more specifically about 15 weight percent. The remainder of the chewable capsule components can vary, generally, between about 2 and about 25 weight percent composed of a maltitol, xylitol, flavoring agent(s), sweetener(s), coloring agent(s), etc. or combination thereof.

The amount of maltitol in the chewable soft gel shell can be varied from zero weight percent, thereby providing a hydrogenated starch hydrolysate free chewable capsule, to about 40 weight percent, more specifically between about 1 and about 4 weight percent more particularly between about 2 and about 3 weight percent.

The amount of xylitol in the chewable soft gel shell can be varied from about 1 to about 20 weight percent, more specifically from about 2 to about 10 weight percent and more particularly about 9 weight percent.

Coloring agents include those listed as being suitable for the fill material and can be formulated in the chewable soft gel shell from about zero weight percent to about 5 weight percent, more specifically between about 0.5 and about 3 weight percent and in particular about 2 weight percent.

Flavoring agents useful in the chewable soft gelatin shell include those that are generally water soluble or water dispersible and can include cinnamon, cinnamon oil, citric acid, lemon oil, orange oil, nutmeg oil, peppermint oil, rose oil, spearmint, spearmint oil, strawberry oil, bacon flavor, barbeque flavors, beef, beef fat, cheese flavors, such as cheddar, nacho, mozzarella, romano, parmesan, chicken, clam, egg, fat, fish, ham, hot dog, lamb, lard, liver, lobster, meat and cheese blend, oyster, pizza, pork, pork liver, prawn, savory, seafood, smoked salmon, steak, taco, tallow and teriyaki and can be formulated in the mixture from about zero weight percent to about 5 weight percent, more specifically between about 0.5 and about 5 weight percent and in particular about 4 weight percent.

Sweeteners include sugars and saccharin and can be present in the chewable soft gel or fill material.

After the capsule is processed, the water content of the final capsule is often between about 5 and about 10 weight percent, more particularly 7 and about 12 weight percent, and more specifically between about 9 and about 10 weight percent.

As for the manufacturing, it is contemplated that standard soft shell gelatin capsule manufacturing techniques can be used to prepare the chewable soft-shell product. Examples of useful manufacturing techniques are the plate process, the rotary die process pioneered by R. P. Scherer, the process using the Norton capsule machine, and the Accogel machine and process developed by Lederle. Each of these processes are mature technologies and are all widely available to any one wishing to prepare soft gelatin capsules.

Typically, when a chewable soft gel capsule is prepared, the total weight is between about 250 milligrams and about 2.5 gram in weight, e.g., 400-750 milligrams. Therefore, the total weight of additives, such as vitamins and antioxidants, is between about 80 milligrams and about 2000 milligrams, alternatively, between about 100 milligrams and about 1500 milligrams, and in particular between about 120 milligrams and about 1200 milligrams.

In one exemplary embodiment, the active agent is dissolved in vitamin E mixed tocopherols, natural mixed carotenoids and yellow to afford the fill material.

In one aspect, the chewable soft gelatin shell can be prepared by combining pork skin gelatin (200-220 bloom), glycerin (99% USP Grade), purified deionized water, D-maltitol syring (minimum 50%), xylitol, cochineal extract (AP Blend 3485), caramel liquid, titanium dioxide and bacon flavored powder.

Alternatively, the chewable soft gelatin shell can be prepared by combining pork skin gelatin, glycerin, water, agar, maltitol syrup, xylitol, cochineal extract, caramel liquid, titanium dioxide and bacon flavored powder.

The present invention also provides packaged formulations of chewable soft gelatin capsule containing the active agent and instructions for its use. Typically, the packaged formulation, is administered to an individual in need thereof that requires the active agent to diminish a disease or condition, or to increase an essential material lacking in the individual's daily dietary regime. Typically, the dosage requirements is between about 1 to about 4 dosages a day.

The following paragraphs enumerated consecutively from 1 through 15 provide for various aspects of the present invention. In one embodiment, in a first paragraph (1), the present invention provides a chewable soft gelatin capsule, comprising a chewable soft gelatin shell that includes gelatin, a plasticizer that is not xylitol, xylitol, water; and a fill material encapsulated within the chewable soft gelatin shell, comprising an active agent and, optionally, an acceptable carrier.

2. The chewable soft gelatin capsule of paragraph 1, wherein the active agent is mixed tocopherols.

3. The chewable soft gelatin shell of either of paragraphs 1 or 2, wherein the plasticizer is glycerin, sorbitol, low molecular weight polyols or mixtures thereof.

4. The chewable soft gelatin shell of any of paragraphs 1 through 3, wherein the plasticizer is glycerin.

5. The chewable soft gelatin shell of any of paragraphs 1 through 4, further comprising a coloring agent.

6. The chewable soft gelatin shell of any of paragraphs 1 through 5, wherein the coloring agent is carmine, caramel, titanium dioxide or mixtures thereof.

7. The chewable soft gelatin shell of any of paragraphs 1 through 6, further comprising a flavoring agent.

8. The fill material of any of paragraphs 1 through 7, further comprising a flavoring agent.

9. The fill material of any of paragraphs 1 through 8, wherein the carrier is rice bran oil or beeswax.

10. The chewable soft gelatin capsule of any of paragraphs 1 through 9, wherein the shell does not contain a hydrogenated starch hydrolysate.

11. The chewable soft gelatin capsule of any of paragraphs 1 through 10, further comprising maltitol in the shell, wherein the maltitol is present in less than about 4 percent by weight of the total weight of the shell composition.

12. The chewable soft gelatin capsule of any of paragraphs 1 through 11, wherein the maltitol is present in an amount between about 1 and about 3 weight percent of the total weight of the shell composition.

13. The chewable soft gelatin capsule of any of paragraphs 1 through 12, wherein the maltitol is present in an amount of about 2 weight percent of the total weight of the shell composition.

14. A method to deliver an active agent, comprising administering to a subject, an active agent encapsulated in a chewable soft gelatin capsule comprising a chewable soft gelatin shell that includes gelatin, a plasticizer that is not xylitol, xylitol, water; and a fill material encapsulated within the chewable soft gelatin shell, including the active agent and, optionally, an acceptable carrier.

15. A packaged pharmaceutical comprising a chewable soft gelatin capsule comprising a chewable soft gelatin shell that includes gelatin, a plasticizer that is not xylitol, xylitol, water, a fill material encapsulated within the chewable soft gelatin shell, comprising an active agent and, optionally, an acceptable carrier; and instructions of use for administration of the active agent.

The following examples are intended to be illustrative only and should not be considered limiting.

### EXAMPLES

Exemplary formulations of soft gelatin capsule materials are as follows:

### Example 1

| Component | Weight |
|---|---|
| Pork skin Gelatin 200-220 Bloom | 34.00 kg |
| Glycerin (99%, USP Grade) | 21.90 kg |
| Purified D.I. Water, USP | 32.75 kg |
| D-Maltitol Syrup, minimum 50% | 10.00 kg |
| Xylitol | 1.5 kg |
| Cochineal Extract AP Blend 3485 | 1.00 kg |
| Caramel liquid | 1.00 kg |
| Titanium Dioxide | 0.15 kg |
| Bacon Flavored Powder | 5.00 kg |

### Example 2

| Component | Weight |
|---|---|
| Pork skin Gelatin | 29.00 kg |
| Glycerin | 20.17 kg |
| Water | 31.50 kg |
| Agar | 2.5 kg |
| Maltitol Syrup | 15.00 kg |
| Xylitol | 1.50 kg |
| Cochineal Extract | 1.00 kg |
| Caramel liquid | 1.00 kg |
| Titanium dioxide | 0.15 kg |
| Bacon Flavored Powder | 5.00 kg |

### Example 3

| Component | Weight |
|---|---|
| Gelatin | 40 g |
| Glycerin | 35 g |
| Purified Water | 17 g |
| Maltitol | 3 g |
| Xylitol | 10 g |
| Flavoring | 5 g |
| Coloring | 2.3 g |

The making of this gelatin for the shell requires adding the water, maltitol, glycerin and xylitol, and heating to between about 50 and about 60° C. until the xylitol is completely melted, then adding the gelatin and heating for one hour, optionally, under vacuum between about 50 and about 60° C. To the heated mixture is added the coloring agent(s) (cochineal, caramel and titanium dioxide) into the gelatin mass (the titanium dioxide was previously mixed with an equal amount of titanium dioxide powder and glycerin, and the amount of glycerin used is represented in the total glycerin in the above formula). At this time the flavoring powder, which was previously dissolved into an equal amount of purified D.I. water (also represented in the total water in the above formula) is added and mixed into the gelatin mass, and the gelatin is placed into receivers that are heated to between about 50 and about 60° C., for between about 4 and about 6 hours.

The shell material can be used at this point to encapsulate the fill material or stored in a heated receiver to keep the shell material flowable.

Although the present invention has been described with reference to preferred embodiments, persons skilled in the art will recognize that changes may be made in form and detail without departing from the spirit and scope of the invention.

All literature and patent references cited throughout the application are incorporated by reference into the application for all purposes.

What is claimed is:

1. A chewable soft gelatin capsule, comprising;
a chewable soft gelatin shell comprising:
gelatin;
a plasticizer that is not xylitol;
xylitol;
water; and
a fill material encapsulated within said chewable soft gelatin shell, comprising an active agent and, optionally, an acceptable carrier.

2. The soft gelatin capsule of claim 1, wherein said active agent is mixed tocopherols.

3. The chewable soft gelatin shell of claim 1, wherein said plasticizer is glycerin, sorbitol, low molecular weight polyols or mixtures thereof.

4. The chewable soft gelatin shell of claim 1, wherein said plasticizer is glycerin.

5. The chewable soft gelatin shell of claim 1, further comprising a coloring agent.

6. The chewable soft gelatin shell of claim 5, wherein said coloring agent is carmine, caramel, titanium dioxide or mixtures thereof.

7. The chewable soft gelatin shell of claim 1, further comprising a flavoring agent.

8. The fill material of claim 1, further comprising a flavoring agent.

9. The fill material of claim 1, wherein said carrier is rice bran oil or beeswax.

10. The chewable soft gelatin capsule of claim 1, wherein said shell does not contain a hydrogenated starch hydrolysate.

11. The chewable soft gelatin capsule of claim 1, further comprising maltitol in said shell, wherein said maltitol is present in less than about 4 percent by weight of the total weight of the shell composition.

12. The chewable soft gelatin capsule of claim 11, wherein said maltitol is present in an amount between about 1 and about 3 weight percent of the total weight of the shell composition.

13. The chewable soft gelatin capsule of claim 11, wherein said maltitol is present in an amount of about 2 weight percent of the total weight of the shell composition.

14. A method to deliver an active agent, comprising
administering to a subject, an active agent encapsulated in a chewable soft gelatin capsule comprising
a chewable soft gelatin shell comprising:
gelatin;
a plasticizer that is not xylitol;
xylitol;
water; and
a fill material encapsulated within said chewable soft gelatin shell, comprising said active agent and, optionally, an acceptable carrier.

15. A packaged pharmaceutical comprising:
a chewable soft gelatin capsule comprising
a chewable soft gelatin shell comprising:
gelatin;
a plasticizer that is not xylitol;
xylitol;
water;
a fill material encapsulated within said chewable soft gelatin shell, comprising
an active agent and, optionally, an acceptable carrier; and
instructions of use for administration of said active agent.
